(19) United States
(12) Patent Application Publication
(10) Pub. No.: US 2011/0274680 A1
(43) Pub. Date: Nov. 10, 2011

(54) Chemical composition and its delivery for lowering the risks of alzheimer's, cardiov ascular and type-2 diabetes diseases

(76) Inventors: Mohammad A. Mazed, Yorba Linda, CA (US); Sayeeda Mazed, Yorba Linda, CA (US)

(21) Appl. No.: 13/135,832

(22) Filed: Jul. 15, 2011

Related U.S. Application Data
(63) Continuation-in-part of application No. 12/573,012, filed on Oct. 2, 2009, now Pat. No. 8,017,147.

(51) Int. Cl. A61K 36/00; A61K 38/43; A61K 31/122; A61P 25/28; A61K 36/752; A61P 9/00; A61P 3/10; A61K 36/82; A61K 38/44

(52) U.S. Cl. 424/94.4; 424/729; 424/94.1; 514/690; 424/725; 424/736

(57) ABSTRACT

Synergistic chemical compositions of bioactive compounds in a dietary supplement for lowering the risks of Alzheimer's, Cardiovascular and Diabetes diseases; chemical compositions of a sugar free super sweetener for people with Type-2 Diabetes disease and a targeted nano delivery of bioactive compounds and/or bioactive molecules are described. Furthermore, microelectro-mechanical system (Mems) based passive and active (based on feedback diagnostics data) delivery systems and methods of bioactive compounds and/or bioactive molecules are also described.

### CROSS REFERENCE OF RELATED APPLICATIONS

The present application is a continuation-in-part (CIP) of and claims priority to U.S. Non-provisional patent application Ser. No. 12/573,012, entitled, “NUTRITIONAL SUPPLEMENT FOR THE PREVENTION OF CARDIOVASCULAR DISEASE, ALZHEIMER'S DISEASE, DIABETES AND REGULATION AND REDUCTION OF BLOOD SUGAR AND INSULIN RESISTANCE”, filed on Oct. 2, 2009.

### FIELD OF THE INVENTION

The present invention relates to synergistic chemical compositions of bioactive compounds in a dietary supplement for lowering the risks of Alzheimer's, Cardiovascular and Diabetes diseases. The present invention relates to chemical compositions of a sugar free sweetener/super sweetener for people with Type-2 Diabetes disease. The present invention also relates to a nano encapsulation and targeted nano delivery of bioactive compounds and/or bioactive molecules for lowering the risks of Alzheimer's, Cardiovascular and Diabetes diseases. Furthermore, the present invention also relates to microelectro-mechanical system (MEMS) based passive and active delivery of bioactive compounds and/or bioactive molecules.

### BACKGROUND OF THE INVENTION

One of the most intriguing discoveries is that many risk factors for Cardiovascular, Type-1 Diabetes and Type-2 Diabetes diseases are risk factors for Alzheimer's disease (also known as Type-3 Diabetes disease).

Studies suggest that high blood cholesterol levels are important risk factors for Alzheimer's disease. If blood flow is restricted because of a buildup of plaque in brain cells, less oxygen gets to the brain and fewer waste residues leave the brain.

Type-1 Diabetes disease is caused by autoimmune destruction of insulin-producing cells in the pancreas, resulting in high blood sugar. The drugs that block Effector Memory T cells, may offer some hope of delaying Type-1 Diabetes disease.

Type-2 Diabetes disease is linked to metabolic syndrome/obesity—hence macrophages in fat tissues. The macrophages in fat tissues produce “cytokine” molecules, which cause inflammations in the pancreas. Such inflammations in the pancreas increase the insulin (a hormone needed to convert carbohydrates, glucose and others foods into energy needed for daily life) resistance. Gradually the pancreas loses its ability to produce insulin.

Diabetes disease is marked by high levels of blood glucose resulting from defects in insulin production and/or inaction. Diabetes disease can lead to serious complications (e.g., kidney disease, high blood pressure, stroke and premature death). But people with Diabetes disease can control/manage the disease and lower the risks of serious complications.

### SUMMARY OF THE INVENTION

The present invention relates to synergistic chemical compositions of bioactive compounds in a dietary supplement for lowering the risks of Alzheimer's, Cardiovascular and Diabetes diseases.

Furthermore, the present invention relates to chemical compositions of a sugar free super sweetener for people with Type-2 Diabetes disease.

Furthermore, the present invention relates to a nano encapsulation and targeted nano delivery of bioactive compounds and/or bioactive molecules for lowering the risks of Alzheimer's, Cardiovascular and Diabetes diseases.

Furthermore, the present invention relates to a microelectro-mechanical system (MEMS) enabled passive delivery of bioactive compounds and/or bioactive molecules.

Furthermore, the present invention relates to a MEMS enabled active (based on feedback diagnostics data) delivery of bioactive compounds and/or bioactive molecules.

### BRIEF DESCRIPTION OF THE TABLES AND FIGURES

The present invention is better understood upon consideration of the description in conjunction with the following tables and drawings.

Table-1 illustrates a synergistic chemical (solid) composition of a dietary supplement for lowering the risks of Alzheimer's disease.

Table-2 illustrates a synergistic chemical (solid) composition of a dietary supplement for lowering the risks of Cardiovascular disease.

Table-3 illustrates a synergistic chemical (solid) composition of a dietary supplement for lowering the risks of Type-2 Diabetes disease.

Table-4 illustrates a synergistic chemical (liquid) composition of a super antioxidant dietary supplement/tonic for lowering the risks of Alzheimer's, Cardiovascular and Type-2 Diabetes diseases.

Table-5 illustrates a synergistic chemical (solid) composition of a sugar free sweetener for people with Type-2 Diabetes disease.

Table-6, Table-7, Table-8, Table-9, Table-10, Table-11, Table-12 Table-13, Table-14, Table-15, Table-16, Table-17 and Table-18 illustrate a synergistic chemical (solid) composition of a sugar free super sweetener for people with Type-2 Diabetes disease.

FIGS. 1A and 1B illustrate interactions of Alzheimer's disease related genes/proteins with a set of bioactive compounds (e.g., an antioxidant/botanical (compound/extract)/enzyme/enzymatic antioxidant/micronutrient (mineral/vitamin)).

FIGS. 2A and 2B illustrate interactions of Type-2 Diabetes disease related genes/proteins with a set of bioactive compounds.

FIGS. 3A, 3B, 3C, 3D and 3E illustrate targeted (with dual targeting ligands) delivery of bioactive compounds and/or bioactive molecules (e.g., GO-Y030, micro-RNA (mi-RNA) and small interfering RNA (si-RNA)) utilizing a nanoshell and a nanocarrier.

FIGS. 4A, 4B, 4C, 4D, 4E, 4F, 4G, 4H, 4I, 4J 4K, 4L and 4M illustrate a passive delivery of bioactive compounds and/or bioactive molecules utilizing a cluster of nano crystals and/or a MEMS reservoir.

FIG. 5 illustrates an active delivery of bioactive compounds and/or bioactive molecules, utilizing a MEMS reservoir and a micropump of Pb(Zr,Ti)O₃ (PZT) material.

FIGS. 6A, 6B and 6C illustrate an integrated 2-D photonics crystal enabled optical diagnostics biomodule to detect a disease specific biomarker.

FIG. 6D illustrates the Stokes Shift (difference in absorption and fluorescence emission wavelengths) due to a disease specific biomarker.

FIGS. 7A, 7B and 7C illustrate an integrated graphene enabled electrical diagnostics biomodule to detect a disease specific biomarker.

FIG. 8A illustrates a bioelectronics subsystem for active (based on feedback diagnostics data) delivery of bioactive compounds and/or bioactive molecules and simultaneous detection of a disease specific biomarker.

FIG. 8B illustrates a real-life application of the above bioelectronics subsystem.

FIG. 9A illustrates a smart retinal contact lens for a passive and/or active delivery of bioactive compounds and/or bioactive molecules.

FIG. 9B illustrates a real-life application of the above smart retinal contact lens.

### DETAIL DESCRIPTION OF THE INVENTION

[Bioactive Compounds &/or Bioactive Molecules Interactions with Genes/Proteins]

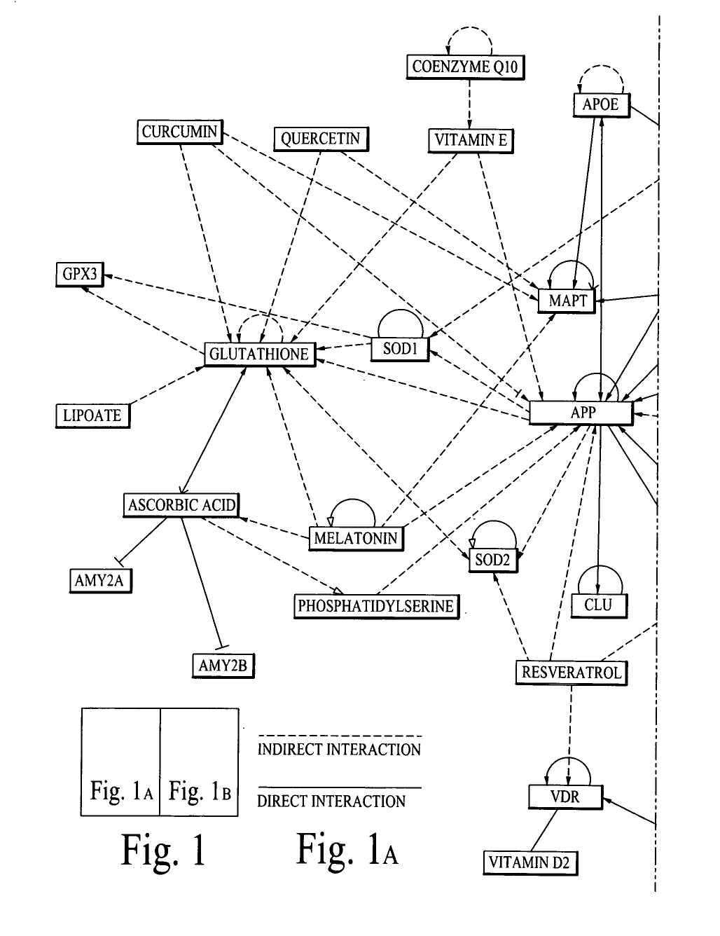

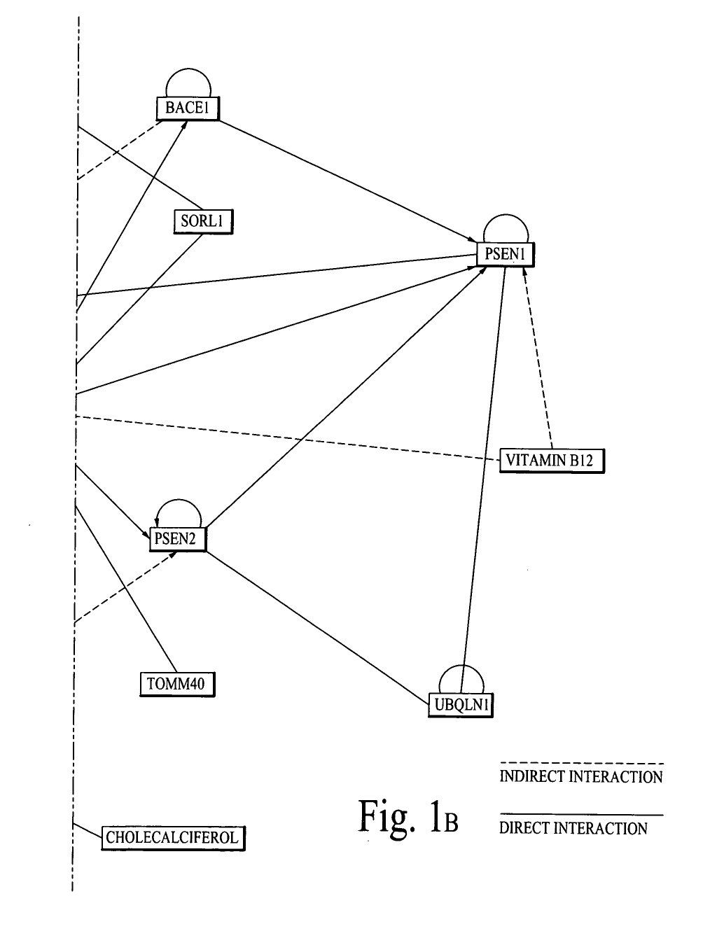

FIGS. 1A and 1B illustrate interactions of Alzheimer's disease related genes/proteins (e.g., APOE, APP, BACE1, CLU, MAPT/TAU, PSEN1, PSEN2, SORL1, TOMM40 and UBQLN1) with bioactive compounds, utilizing a comprehensive biological pathway analysis software.

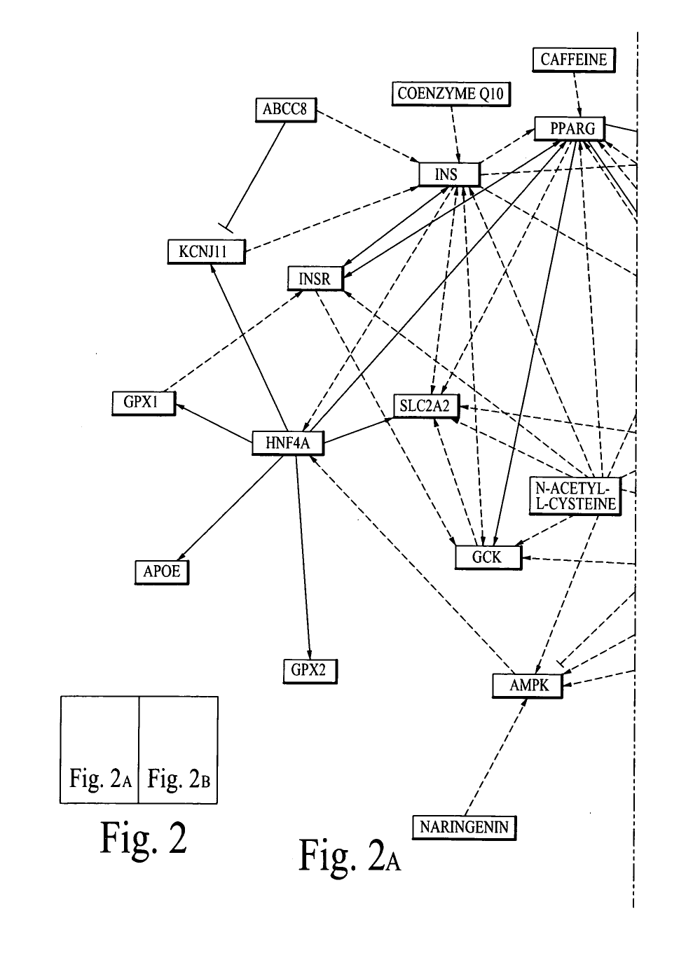

FIGS. 2A and 2B illustrate interactions of Type-2 Diabetes disease related genes/proteins (e.g., ABCC8, GCK, HNF4A, INS, INSR, KCNJ11, LPL, PPARG and SLC2A2) with bioactive compounds, utilizing a comprehensive biological pathway analysis software.

Furthermore, from the analysis of the above, it was found that Alzheimer's disease related gene/protein APOE is also linked with Type-2 Diabetes disease related gene/protein HNF4A.

FIGS. 1A, 1B, 2A and 2B are critical to design chemical compositions of dietary supplements for lowering the risks of Alzheimer's and Diabetes diseases.

### [Compositions]

TABLE 1
Solid (~2 Softgels) Composition For Lowering
The Risks of Alzheimer's Disease

| | Unit | +/−50% | WT % |
|---|---|---|---|
| Botanicals | | | |
| Bacopa monnieri⁺ | Mg | 200 | 3.16% |
| Camellia sinensis⁺ (Black) | Mg | 200 | 3.16% |
| Camellia sinensis⁺ (Green) | Mg | 200 | 3.16% |
| Camellia sinensis⁺ (White) | Mg | 200 | 3.16% |
| Cinnamomum zeylanicum⁺ | Mg | 200 | 3.16% |
| Curcuma longa⁺ (Or Curcumin | Mg | 1000 | 15.80% |
| Derived Molecules) | | | |
| Evolvulus alsinoide⁺ | Mg | 200 | 3.16% |
| Hypericum perforatum⁺ | Mg | 200 | 3.16% |
| Mucuna pruriens⁺ | Mg | 200 | 3.16% |
| Nigella sativa/kalonji⁺ | Mg | 200 | 3.16% |
| Paeoniae alba⁺ | Mg | 200 | 3.16% |
| Salvia miltiorrhiza⁺ | Mg | 200 | 3.16% |
| Withania somnifera⁺ | Mg | 200 | 3.16% |
| Chemicals | | | |
| Acetyl-L-Carnitine | Mg | 200 | 3.16% |
| Alpha-R-Lipoic Acid | Mg | 20 | 0.32% |
| Caffeine | Mg | 20 | 0.32% |
| Citicoline | Mg | 200 | 3.16% |
| Coenzyme Q₁₀ (From Ubiquinol) | Mg | 200 | 3.16% |
| Creatine | Mg | 550 | 8.70% |
| D-Ribose (Nano Sized) | Mg | 200 | 3.16% |
| L-Glutathione (Or Ebselen Or N- | Mg | 200 | 3.16% |
| Acetyl-L-Cysteine) | | | |
| L-Theanine | Mg | 200 | 3.16% |
| Melatonin | Mg | 5 | 0.08% |
| Pterostilbene | Mg | 200 | 3.16% |
| Quercetin (Nano Encapsulated) | Mg | 200 | 3.16% |
| Resveratrol (Nano Encapsulated) | Mg | 200 | 3.16% |
| Superoxide Dismutase (SOD)* | Mg | 200 | 3.16% |
| (Nano Encapsulated) | | | |
| Uric Acid (From Inosine: Hypoxanthine | Mg | 200 | 3.16% |
| Ribose) | | | |
| Vitamins | | | |
| Vitamin B₃ (Nicotinamide) | Mg | 125 | 1.98% |
| Vitamin B₆ (Pyridoxine | Mg | 1.5 | 0.02% |
| Alpha-Ketoglutarate) | | | |
| Vitamin B₇ | Mg | 0.25 | 0.00% |
| Vitamin B₉ | Mg | 0.5 | 0.01% |
| Vitamin B₁₂ (Methylcobalamin) | Mg | 0.07 | 0.00% |
| Vitamin D₃ | IU/Mg | 350/0.008 | 0.00% |
| Minerals | | | |
| Lithium Orotate | Mg | 5 | 0.08% |
| Selenium (Selenomethionine) | Mg | 0.1 | 0.00% |
| Zinc (L-Opti) | Mg | 5 | 0.08% |
| Total Weight | G | ~6.5 | 100.00% |

The above Table-1 can include a botanical mixture of: Aronia melanocarpa⁺, Citrus limonum⁺, Daucus carota⁺, Hibiscus spp.⁺, Malus domestica⁺, Ribes nigrum⁺, Sambucus nigra⁺ and Vaccinium spp.⁺

TABLE 2
Solid (~2 Softgels) Composition For Lowering
The Risks Of Cardiovascular Disease

| | Unit | +/−50% | WT % |
|---|---|---|---|
| Botanicals | | | |
| Crataegus oxyacantha⁺ | Mg | 200 | 2.65% |
| Inula racemosa⁺ | Mg | 200 | 2.65% |
| Irvingia gabonensis⁺ | Mg | 200 | 2.65% |
| Terminalia arjuna⁺ | Mg | 200 | 2.65% |
| Chemicals | | | |
| L-Glutathione (Or Ebselen Or | Mg | 200 | 2.65% |
| N-Acetyl-L-Cysteine) | | | |
| Plant Sterols | Mg | 5000 | 66.23% |
| Pterostilbene | Mg | 200 | 2.65% |
| Quercetin (Nano Encapsulated) | Mg | 200 | 2.65% |
| Resveratrol (Nano Encapsulated) | Mg | 200 | 2.65% |
| Superoxide Dismutase (SOD)* | Mg | 200 | 2.65% |
| (Nano Encapsulated) | | | |
| Vitamins | | | |
| Vitamin A (Beta Carotene) | IU/Mg | 1000/0.3 | 0.00% |
| Vitamin B₃ (Nicotinamide) | Mg | 125 | 1.66% |
| Vitamin B₆ (Pyridoxine Alpha- | Mg | 1.5 | 0.02% |
| Ketoglutarate) | | | |
| Vitamin B₇ | Mg | 0.25 | 0.00% |
| Vitamin B₉ | Mg | 0.5 | 0.01% |
| Vitamin B₁₂ (Methylcobalamin) | Mg | 0.07 | 0.00% |
| Vitamin C | Mg | 200 | 2.65% |
| Vitamin E (Natural) | IU/Mg | 25/16.75 | 0.22% |
| Minerals | | | |
| Magnesium | Mg | 200 | 2.65% |
| Potassium | Mg | 200 | 2.65% |
| Selenium (Selenomethionine) | Mg | 0.1 | 0.00% |
| Zinc (L-Opti) | Mg | 5 | 0.07% |
| Total Weight | G | ~7.5 | 100.00% |

The above Table-2 can include a botanical mixture of: Aronia melanocarpa⁺, Citrus limonum⁺, Daucus carota⁺, Hibiscus spp.⁺, Malus domestica⁺, Ribes nigrum⁺, Sambucus nigra⁺ and Vaccinium spp.⁺

TABLE 3
Solid (~2 Softgels) Composition For Lowering
The Risks Of Type-2 Diabetes Disease

| | Unit | +/−50% | WT % |
|---|---|---|---|
| Botanicals | | | |
| Andrographis paniculata⁺ | Mg | 200 | 2.00% |
| Artemisia princeps⁺ | Mg | 200 | 2.00% |
| Camellia sinensis⁺ (Black) | Mg | 200 | 2.00% |
| Camellia sinensis⁺ (Green) | Mg | 200 | 2.00% |
| Camellia sinensis⁺ (White) | Mg | 200 | 2.00% |
| Cinnamomum zeylanicum⁺ | Mg | 200 | 2.00% |
| Coccinia indica⁺ | Mg | 750 | 7.51% |
| Euterpe oleracea⁺ | Mg | 200 | 2.00% |
| Hippophae rhamnoides⁺ | Mg | 200 | 2.00% |
| Irvingia gabonensis⁺ | Mg | 200 | 2.00% |
| Lycium barbarum⁺ | Mg | 200 | 2.00% |
| Momordica charantia⁺ | Mg | 200 | 2.00% |
| Nigella sativa/kalonji⁺ | Mg | 200 | 2.00% |
| Phyllanthus emblica⁺ | Mg | 200 | 2.00% |
| Punica granatum⁺ | Mg | 200 | 2.00% |
| Salacia oblonga⁺ | Mg | 750 | 7.51% |
| Vitis spp.⁺ | Mg | 200 | 2.00% |
| Botanical Mixture | | | |
| Aronia melanocarpa⁺, Citrus | Mg | 1000 | 10.02% |
| limonum⁺, Daucus carota⁺, | | | |
| Hibiscus spp.⁺, Malus domestica⁺, | | | |
| Ribes nigrum⁺, Sambucus nigra⁺ | | | |
| and Vaccinium spp.⁺ - each is about | | | |
| 12.5% of the total weight | | | |
| Chemicals | | | |
| Acetyl-L-Carnitine | Mg | 200 | 2.00% |
| Alpha-R-Lipoic Acid | Mg | 20 | 0.20% |
| Beta Glucan | Mg | 200 | 2.00% |
| Coenzyme Q₁₀ (From Ubiquinol) | Mg | 200 | 2.00% |
| D-Ribose (Nano Sized) | Mg | 750 | 7.51% |
| Epigallocatechin Gallate | Mg | 200 | 2.00% |
| L-Glutathione (Or Ebselen Or | Mg | 200 | 2.00% |
| N-Acetyl-L-Cysteine) | | | |
| Lutein | Mg | 15 | 0.15% |
| Lycopene | Mg | 200 | 2.00% |
| Nobiletin (Or 2000 Mg Naringenin) | Mg | 200 | 2.00% |
| Pterostilbene | Mg | 200 | 2.00% |
| Quercetin (Nano Encapsulated) | Mg | 200 | 2.00% |
| Resveratrol (Nano Encapsulated) | Mg | 200 | 2.00% |
| Sulforaphane | Mg | 200 | 2.00% |
| Superoxide Dismutase (SOD)* (Nano | Mg | 200 | 2.00% |
| Encapsulated) | | | |
| Touchi | Mg | 1000 | 10.02% |
| Vitamins | | | |
| Vitamin A (Beta Carotene) | IU/Mg | 1000/0.3 | 0.00% |
| Vitamin B₃ (Nicotinamide) | Mg | 125 | 1.25% |
| Vitamin B₆ (Pyridoxine Alpha- | Mg | 1.5 | 0.02% |
| Ketoglutarate) | | | |
| Vitamin B₇ | Mg | 0.25 | 0.00% |
| Vitamin B₉ | Mg | 0.5 | 0.01% |
| Vitamin B₁₂ (Methylcobalamin) | Mg | 0.07 | 0.00% |
| Vitamin C | Mg | 100 | 1.00% |
| Vitamin D₃ | IU/Mg | 350/0.008 | 0.00% |
| Vitamin E (Natural) | IU/Mg | 25/16.75 | 0.17% |
| Vitamin K₁ | Mg | 0.025 | 0.00% |
| Vitamin K₂ | Mg | 0.025 | 0.00% |
| Minerals | | | |
| Boron | Mg | 0.15 | 0.00% |
| Calcium | Mg | 50 | 0.50% |
| Chromium Picolinate | Mg | 0.025 | 0.00% |
| Selenium (Selenomethionine) | Mg | 0.1 | 0.00% |
| Zinc (L-Opti) | Mg | 5 | 0.05% |
| Total Weight | G | ~10 | 100% |

TABLE 4
Liquid (~14 Tablespoons) Composition For Super Antioxidant Tonic

| | Unit | +/−50% |
|---|---|---|
| Botanicals | | |
| Actinidia chinenesis⁺ | G | 25 |
| Ananas comosus⁺ | G | 25 |
| Cocos nucifera⁺ | G | 350 |
| Garcinia mangostana⁺ | G | 25 |
| Litchi chinensis⁺ | G | 25 |
| Vitis spp.⁺ | G | 0.75 |
| Botanical Mixture | | |
| Aronia melanocarpa⁺, Citrus limonum⁺, Daucus carota⁺, | G | 25.0 |
| Hibiscus spp.⁺, Malus domestica⁺, Ribes nigrum⁺, | | |
| Sambucus nigra⁺ and Vaccinium spp. ⁺ - each is about | | |
| 12.5% of the total weight | | |
| Chemicals | | |
| Citicoline | G | 0.75 |
| Coenzyme Q₁₀ (From Ubiquinol) | G | 0.75 |
| D-Ribose (Nano Sized) | G | 0.75 |
| L-Analyl-L-Glutamine | G | 0.75 |
| L-Glutathione (Or Ebselen Or N-Acetyl-L-Cysteine) | G | 0.75 |
| L-Theanine | G | 0.75 |
| Plant Sterols | G | 10 |
| Pterostilbene | G | 0.5 |
| Quercetin (Nano Encapsulated) | G | 0.5 |
| Resveratrol (Nano Encapsulated) | G | 0.5 |
| Superoxide Dismutase (SOD)* (Nano Encapsulated) | G | 0.5 |
| Vitamin | | |
| Vitamin C | G | 0.5 |
| Mineral | | |
| Potassium | G | 0.5 |
| Total Weight | G | ~500 |

*present in Citrullus vulgaris⁺

TABLE 5
Solid (Powder) Composition For Sugar Free Sweetener

| Botanicals | Unit | +/−50% |
|---|---|---|
| Erythritol | Mg | 4500 |
| Stevia rebaudiana⁺ | Mg | 20 |
| Total Weight | G | ~4.5 |

TABLE 6
Solid (Powder) Composition For Sugar Free Super Sweetener

| Botanicals | Unit | +/−50% |
|---|---|---|
| Capparis masaikai⁺ (Mabinlins Protein) | Mg | 5 |
| Erythritol | Mg | 4500 |
| Stevia rebaudiana⁺ | Mg | 20 |
| Total Weight | G | ~4.5 |

TABLE 7
Solid (Powder) Composition For Sugar Free Super Sweetener

| Botanicals | Unit | +/−50% |
|---|---|---|
| Curculigo latifolia⁺ (Curculin Protein) | Mg | 5 |
| Erythritol | Mg | 4500 |
| Stevia rebaudiana⁺ | Mg | 20 |
| Total Weight | G | ~4.5 |

TABLE 8
Solid (Powder) Composition For Sugar Free Super Sweetener

| Botanicals | Unit | +/−50% |
|---|---|---|
| Dioscoreophyllum cumminsii⁺ (Monellin Protein) | Mg | 2 |
| Erythritol | Mg | 4500 |
| Stevia rebaudiana⁺ | Mg | 20 |
| Total Weight | G | ~4.5 |

TABLE 9
Solid (Powder) Composition For Sugar Free Super Sweetener

| Botanicals | Unit | +/−50% |
|---|---|---|
| Erythritol | Mg | 4500 |
| Momordica grosvenorii/Siraitia grosvenorii⁺ | Mg | 5 |
| Stevia rebaudiana⁺ | Mg | 20 |
| Total Weight | G | ~4.5 |

TABLE 10
Solid (Powder) Composition For Sugar Free Super Sweetener

| Botanicals | Unit | +/−50% |
|---|---|---|
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Stevia rebaudiana⁺ | Mg | 20 |
| Total Weight | G | ~4.5 |

TABLE 11
Solid (Powder) Composition For Sugar Free Super Sweetener

| Botanicals | Unit | +/−50% |
|---|---|---|
| Erythritol | Mg | 4500 |
| Stevia rebaudiana⁺ | Mg | 20 |
| Synsepalum dulcificum⁺ (Miraculin Protein) | Mg | 5 |
| Total Weight | G | |

TABLE 12
Solid (Powder) Composition For Sugar Free Super Sweetener

| Botanicals | Unit | +/−50% |
|---|---|---|
| Erythritol | Mg | 4500 |
| Stevia rebaudiana⁺ | Mg | 20 |
| Thaumatococcus daniellii⁺ (Thaumatin Protein) | Mg | 1 |
| Total Weight | G | |

TABLE 13
Solid (Powder) Composition For Sugar Free Super Sweetener

| Botanicals | Unit | +/−50% |
|---|---|---|
| Dioscoreophyllum cumminsii⁺ (Monellin Protein) | Mg | 2 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Stevia rebaudiana⁺ | Mg | 20 |
| Total Weight | G | ~4.5 |

TABLE 14
Solid (Powder) Composition For Sugar Free Super Sweetener

| Botanicals | Unit | +/−50% |
|---|---|---|
| Dioscoreophyllum cumminsii⁺ (Monellin Protein) | Mg | 2 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Synsepalum dulcificum⁺ (Miraculin Protein) | Mg | 5 |
| Stevia rebaudiana⁺ | Mg | 20 |
| Total Weight | G | ~4.5 |

TABLE 15
Solid (Powder) Composition For Sugar Free Super Sweetener

| Botanicals | Unit | +/−50% |
|---|---|---|
| Capparis masaikai⁺ (Mabinlins Protein) | Mg | 5 |
| Dioscoreophyllum cumminsii⁺ (Monellin Protein) | Mg | 2 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Synsepalum dulcificum⁺ (Miraculin Protein) | Mg | 5 |
| Stevia rebaudiana⁺ | Mg | 20 |
| Total Weight | G | ~4.5 |

TABLE 16
Solid (Powder) Composition For Sugar Free Super Sweetener

| Botanicals | Unit | +/−50% |
|---|---|---|
| Curculigo latifolia⁺ (Curculin Protein) | Mg | 1 |
| Dioscoreophyllum cumminsii⁺ (Monellin Protein) | Mg | 2 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Synsepalum dulcificum⁺ (Miraculin Protein) | Mg | 5 |
| Stevia rebaudiana⁺ | Mg | 20 |
| Total Weight | G | ~4.5 |

TABLE 17
Solid (Powder) Composition For Sugar Free Super Sweetener

| Botanicals | Unit | +/−50% |
|---|---|---|
| Capparis masaikai⁺ (Mabinlins Protein) | Mg | 1 |
| Curculigo latifolia⁺ (Curculin Protein) | Mg | 1 |
| Dioscoreophyllum cumminsii⁺ (Monellin Protein) | Mg | 2 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Synsepalum dulcificum⁺ (Miraculin Protein) | Mg | 5 |
| Stevia rebaudiana⁺ | Mg | 20 |
| Total Weight | G | ~4.5 |

TABLE 18
Solid (Powder) Composition For Sugar Free Super Sweetener

| Botanicals | Unit | +/−50% |
|---|---|---|
| Capparis masaikai⁺ (Mabinlins Protein) | Mg | 1 |
| Curculigo latifolia⁺ (Curculin Protein) | Mg | 1 |
| Dioscoreophyllum cumminsii⁺ (Monellin Protein) | Mg | 5 |
| Erythritol | Mg | 4500 |
| Pentadiplandra brazzeana⁺ (Brazzein Protein) | Mg | 5 |
| Pentadiplandra brazzeana⁺ (Pentadin Protein) | Mg | 5 |
| Synsepalum dulcificum⁺ (Miraculin Protein) | Mg | 5 |
| Stevia rebaudiana⁺ | Mg | 20 |
| Total Weight | G | ~4.5 |

⁺means extract from any part of the plant

### [Targeted Delivery of Nano Encapsulated Bioactive Compounds &/or Bioactive Molecules]

Bioactive compounds and/or bioactive molecules usually get destroyed by acids/enzymes in the digestive system and only a tiny fraction of the bioactive compounds and/or bioactive molecules are absorbed in the blood stream.

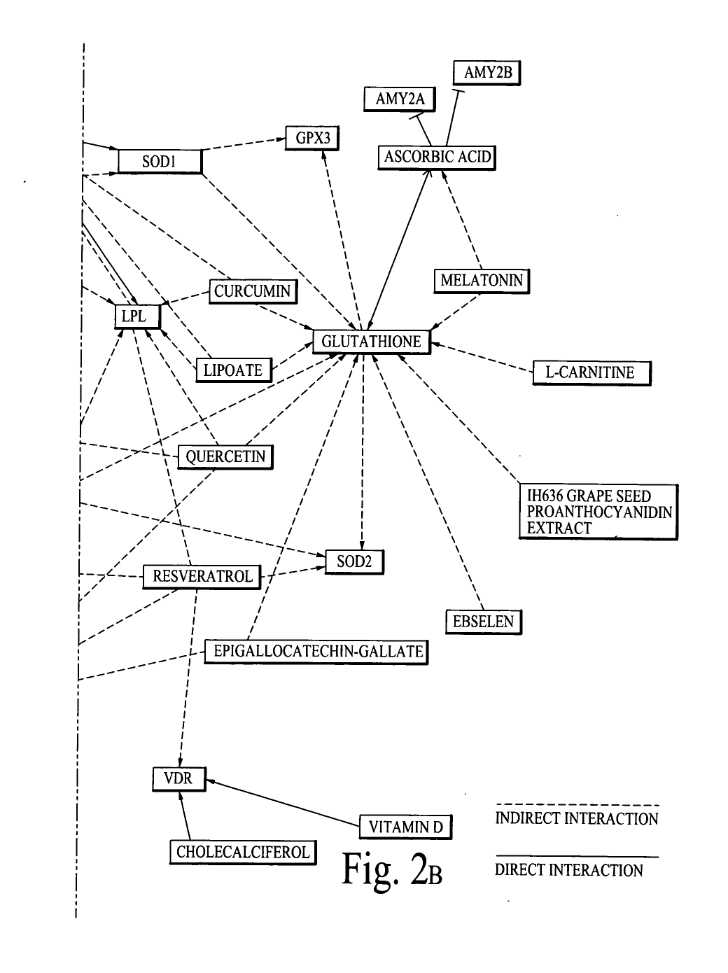

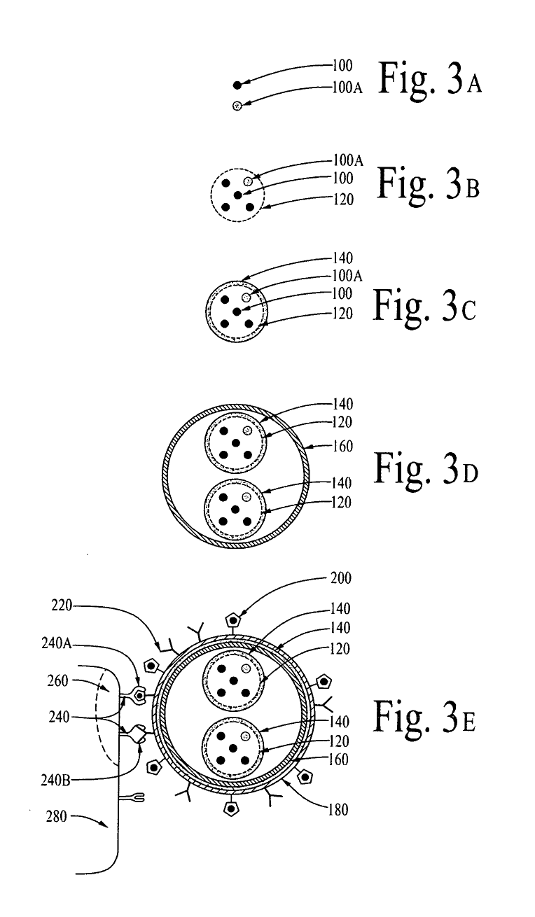

FIG. 3A illustrates a bioactive compound 100 and a bioactive molecule 100A respectively.

FIG. 3B illustrates the bioactive compound 100 and bioactive molecule 100A, which are encapsulated/caged in a non-toxic semi-porous nanoshell (e.g., a cubisome/liposome/liposome synthesized with porous silica particle/nano crystal (e.g., nano diamond/nano Hydroxyapatite)/self-assembling peptide (or protein)/single-domain antibody/synthasome/zein-plant protein)) 120.

Hydroxyapatite is a form of calcium phosphate with a chemical formula Ca₁₀(PO₄)₆(OH)₂.

Synthasome is a spherical hollow nanoshell. It contains an aqueous solution for protecting the bioactive compound 100 and/or bioactive molecule 100A. The synthasome has nano sized channels (e.g., a transmembrane protein) to permit or deny transport of a substance across the synthasome membrane. Furthermore, use of any synthetic polymer material to manufacture synthasome can enable to customize the characteristics (e.g., control permeability, release rate and stability) of the synthasome membrane.

Other nanoshells are dendrimer, ethosome, glycosome, noisome and polymeric micelle.

The interior surface of the nanoshell 120 can be electrically charged (e.g., the interior surface of the nanoshell can have an opposite electrical charge polarity with respect to the electrical charge polarity of the bioactive compound 100 and/or the bioactive molecule 100A encapsulated/caged in the nanoshell 120) to increase the encapsulation efficiency of the bioactive compound 100 and/or bioactive molecule 100A.

The exterior surface of the nanoshell 120 can also be electrically charged to increase the delivery efficiency of the bioactive compound 100 and/or bioactive molecule 100A.

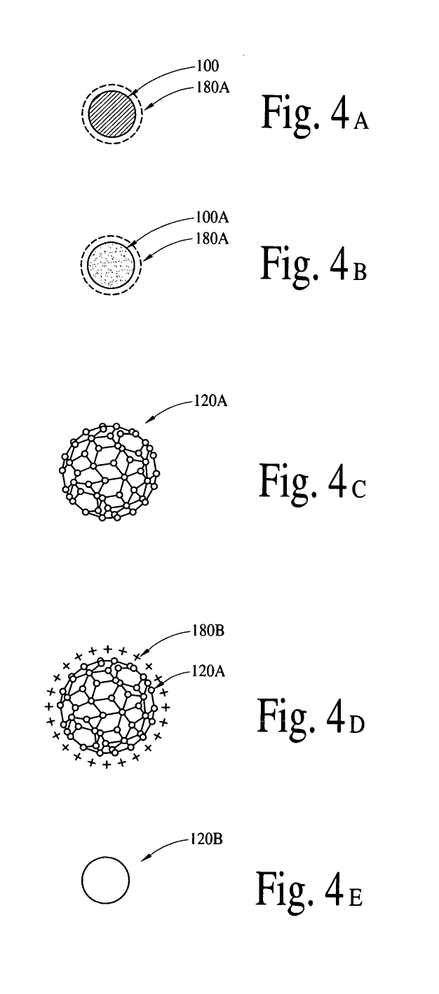

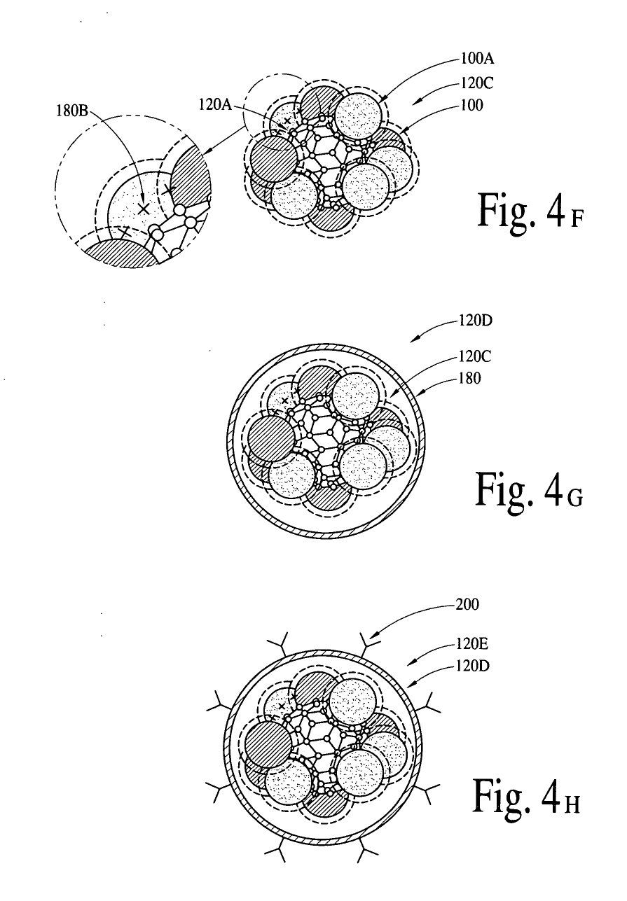

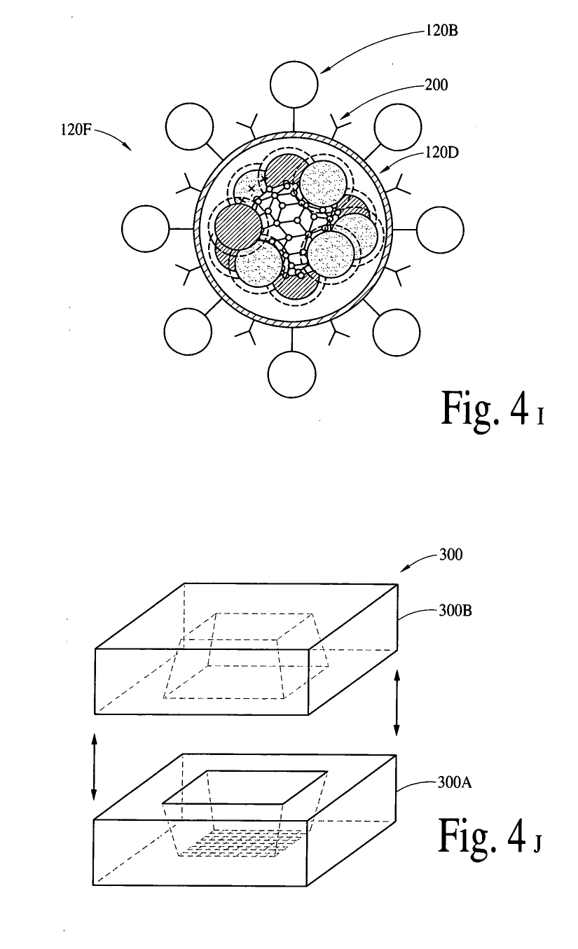

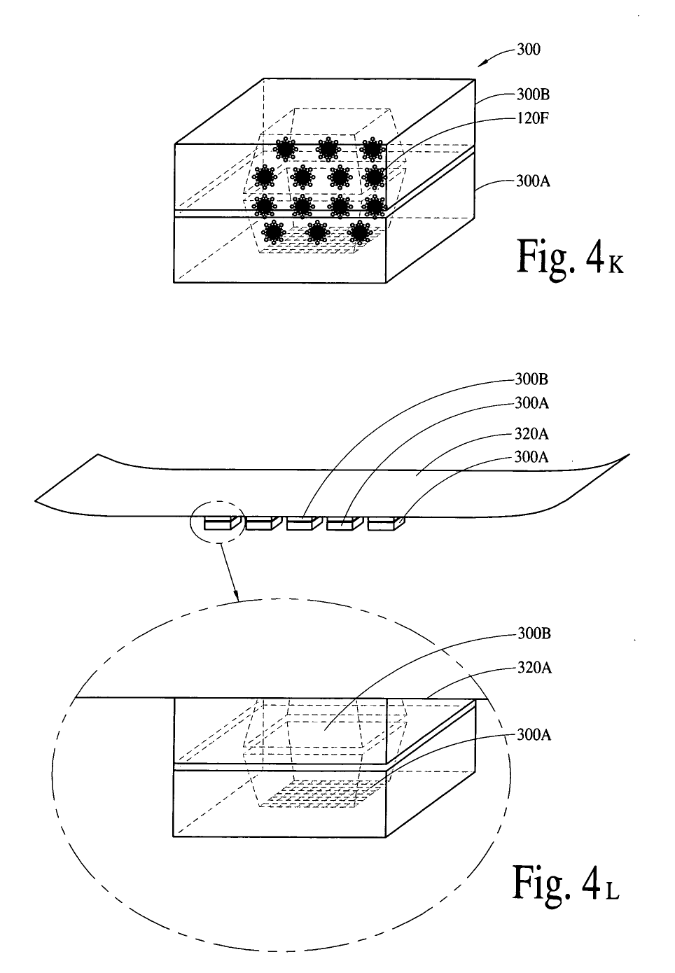

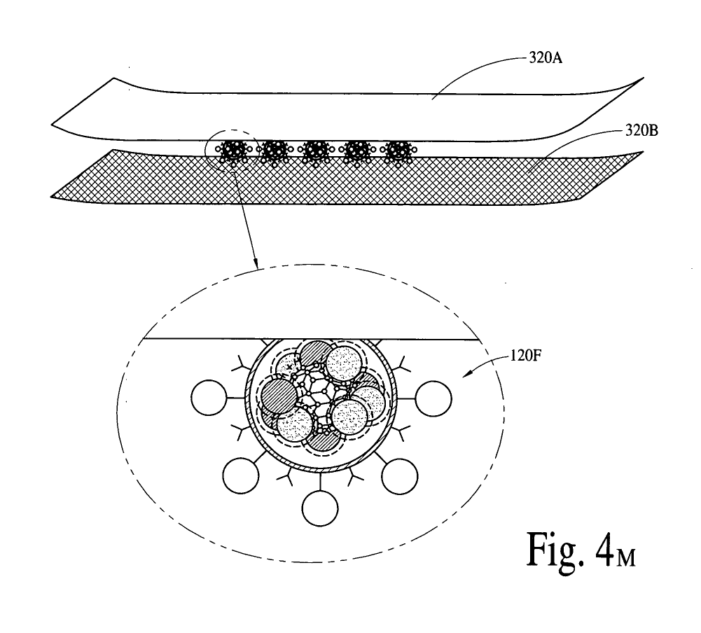

Optionally a fluorophore (preferably a quantum dot/3-D photonic crystal) (e.g., 120B as in FIG. 4E) can be attached to the nanoshell 120 to visualize the delivery of the bioactive compound 100 and/or bioactive molecule 100A.

FIG. 3C illustrates the surface of the nanoshell 120, which is coated with a functional surface (e.g., casein—a milk protein) 140.

FIG. 3D illustrates the functional surface coated nanoshells 120, which are further encapsulated/caged in a nanocarrier (e.g., a natural biopolymer chitosan or a capsosome) 160.

To construct a capsosome, a polymer film is deposited onto small silica spheres. This polymer film is modified with cholesterol. Liposomes (coated with a functional surface (e.g., polyethylene glycol molecule) to shield from the body's immune surveillance and a targeting ligand to deliver encapsulated/caged with the bioactive compound 100 and/or bioactive molecule 100A at specific cells) are anchored to the cholesterol. Subsequently, more polymer films are added and cross-linked by disulfide bridges. Finally, the small silica spheres are finally etched away.

FIG. 3E illustrates the nanocarrier 160, which is also coated with the functional surface (e.g., casein—a milk protein) 140.

The functional surface 140 protects both the nanoshell 120 and the nanocarrier 160 from acids/enzymes in the digestive system.

Another functional surface (e.g., polyethylene glycol molecule) 180 on the nanocarrier 160 shields the nanocarrier 160 from the body's inherent immune surveillance.

Optionally a fluorophore (preferably a quantum dot/3-D photonic crystal) (e.g., 120B as in FIG. 4E) can be attached to the functional surface 180 to visualize the delivery of the bioactive compound 100 and/or bioactive molecule 100A.

A targeting ligand-cobalamin/vitamin B₁₂ 200 (on functional surface 180 of the nanocarrier 160) recognizes and attaches/locks onto the specific types of biological receptors 240A on the intestinal cells 260 (of the small intestinal walls 280).

Another targeting ligand-antibody 220 (on functional surface 180 of the nanocarrier 160) recognizes and attaches/locks onto the specific types of biological receptors 240B on the intestinal cells 260.

Both targeting ligands—(a) cobalamin/vitamin B₁₂ 200 and (b) an antibody 220 (on the functional surface 180 of the nanocarrier 160) act as dual navigators, guiding the nanocarrier 160 to the intestinal cells 260.

The nanocarrier 160 reaches and sticks to the intestinal cells 260, having the biological receptors 240, specifically 240A and 240B on the intestinal cells 260.

Both the nanocarrier 160 and the nanoshell 120, eventually breaks under an external condition (e.g., pH), allowing the bioactive compound 100 and/or bioactive molecule 100A to leak out from the nanoshell 120 into the intestinal cells 260, so that the bioactive compound 100 and/or bioactive molecule 100A can be absorbed in a controlled manner for a longer period of time in the blood stream.

Optionally, the nanoshell 120, (integrated with the functional surface 140, the functional surface (e.g., polyethylene glycol molecule) 180 to shield from the body's inherent immune surveillance, an optional fluorophore and the targeting ligands—(a) cobalamin/vitamin B₁₂ 200 and (b) the antibody 220) can be directly utilized instead of the nanocarrier 160. The nanoshell 120, eventually breaks under an external condition (e.g., pH), allowing the bioactive compound 100 and/or the bioactive molecule 100A to leak out from the nanoshell 120 into the intestinal cells 260, so that the bioactive compound 100 and/or bioactive molecule 100A can be absorbed in a controlled manner for a longer period of time in the blood stream.

### [Passive Delivery of Bioactive Compounds &/or Bioactive Molecules]

FIG. 4A illustrates an expanded view of a negative electrical charged surface 180A on the bioactive compound (e.g., superoxide dismutase (SOD)) 100.

FIG. 4B illustrates an expanded view of a negative electrical charged surface 180A on the bioactive molecule 100A.

FIG. 4C illustrates an expanded view of a (also non-toxic) nano crystal 120A (e.g., a nano diamonds/nano-Hydroxyapatite (HAP)/single-domain antibody). HAP is a form of calcium phosphate with chemical formula Ca₁₀(PO₄)₆(OH)₂.

FIG. 4D illustrates expanded view of a positive electrical charged surface 180B on the nano crystal 120A.

This charge conjugation is optional, but it increases both the encapsulation and delivery efficiency of the bioactive compounds 100 and/or bioactive molecules 100A.

FIG. 4E illustrates an expanded view of a fluorophore (preferably a quantum dot/3-D photonic crystal) 120B.

FIG. 4F illustrates 120C, wherein the negative electrical charged bioactive compounds 100 and/or bioactive molecules 100A are surrounded by a cluster of the positive electrical charged nano crystals 120A.

For example, the above nano assembly 4F with curcumin and curcumin-derived synthetic molecules (FLLL-11, FLLL-12, GO-Y030 and GO-Y031) can be an effective therapy against cancer (including brain cancer).

For example, the above nano assembly 4F with acetyl-L-carnitine, curcumin, curcumin-derived synthetic molecules (FLLL-11, FLLL-12, GO-Y030 and GO-Y031), L-DOPA and melatonin can cross blood brain barrier and can be an effective therapy against neurodegenerative diseases (e.g., Alzheimer's and/or Parkinson's diseases).

For example, the above nano assembly 4F with luric acid can be an effective therapy against acne.

For example, the above nano assembly 4F with nitrous oxide can be an effective therapy against erectile dysfunction.

For example, the above nano assembly 4F with micro-RNA (mi-RNA) or small interfering RNA (si-RNA) can be an effective therapy against various diseases.

FIG. 4G illustrates 120D—wherein 120C is chemically bonded with a functional surface 180. The functional surface 180 shields 120C from the body's inherent immune surveillance.

FIG. 4H illustrates 120E—wherein 120D is chemically bonded with at least one targeting ligand 200. The targeting ligand 200 attaches/locks onto specific types of biological receptors of a specific cell.

FIG. 4I illustrates 120F—wherein 120F is chemically bonded with the fluorophore 120B.

The above nano assembly 4I can be utilized for controlled delivery of the bioactive compounds 100 and/or bioactive molecules 100A over a longer period of time.

FIG. 4J illustrates a MEMS reservoir 300 (e.g., fabricated from silicon/SU-8 resin/liquid crystal polymers/parylene/polyimide material).

The top surface of the MEMS reservoir 300 is 300B.

The bottom surface of the MEMS reservoir 300 is 300A. 300A is semi-porous for sustainable and/or controlled delivery of the bioactive compounds 100 and/or bioactive molecules 100A.

FIG. 4K illustrates 120Fs, which are inserted/caged in the MEMS reservoir 300.

FIG. 4L illustrates the top surface 300B of the MEMS reservoir 300 (with 120F inserted/caged in the MEMS reservoir 300) is attached to an adhesive thin-film 320A as a long-term micro patch (about 15 mm² in area) for sustainable and/or controlled delivery of the bioactive compounds 100 and/or bioactive molecules 100A.

The bottom semi-porous surface of the MEMS reservoir 300 is 300A. 300A is attached onto a transport medium (e.g., a tissue/skin).

FIG. 4M illustrates 120F bonded directly between a non-porous adhesive thin-film 320A and a semi-porous adhesive thin-film 320B as a short-term micro patch (about 15 mm² in area) for sustainable and/or controlled delivery of the bioactive compounds 100 and/or bioactive molecules 100A.

Applications of the above 4M are delivery of any drug (e.g., acetyl-L-carnitine, antibiotics, insulin, L-DOPA, luric acid, melatonin and nitrous oxide).

The semi-porous adhesive thin-film 320B is attached onto the transport medium.

Such a passive delivery of the bioactive compounds 100 and/or bioactive molecules 100A is typically limited by a low permeability of the bioactive compounds 100 and/or bioactive molecules 100A in the transport medium.

### [Active Delivery of Bioactive Compounds &/or Bioactive Molecules]

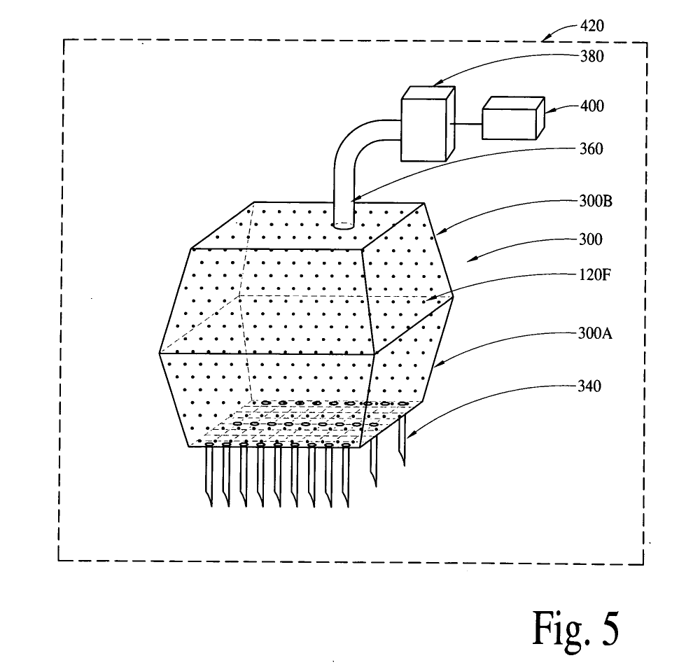

FIG. 5 illustrates the MEMS reservoir 300 (with 120Fs are dispersed in a liquid medium, where 120Fs are encapsulating/caging the bioactive compounds 100 and/or bioactive molecules 100A) of about 1 mm total thickness, monolithically integrated with an array of microneedles 340 of a biocompatible material (e.g., silicon/SU-8 resin/liquid crystal polymers/parylene/polyimide) at the bottom surface 300A of the MEMS reservoir 300.

The microneedles 340 are about 450 micron long with an internal hole-diameter of about 45 micron.

The MEMS reservoir 300 is connected to a microflow tube 360, which is further connected to a micropump 380; the micropump 380 is powered by an electrical power component 400.

Such a MEMS biomodule configuration 420 is utilized to achieve a higher permeability through the transport medium for delivering bioactive compounds 100 and/or bioactive molecules 100A.

### [Optical Diagnostics Module for Detection of a Disease Specific Biomarker]

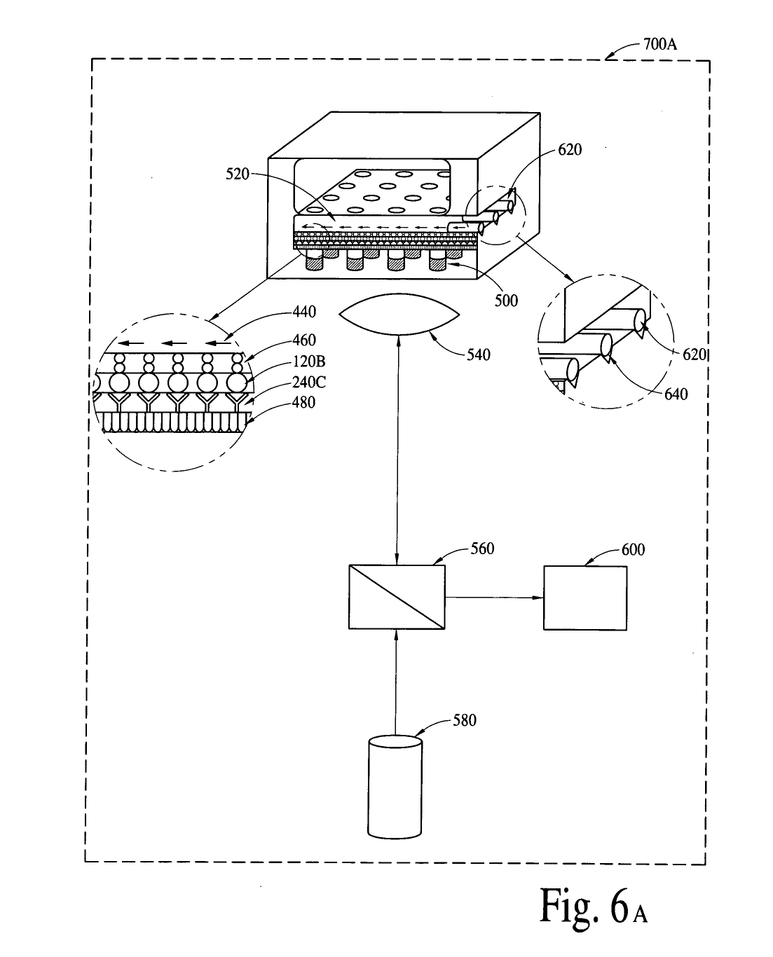

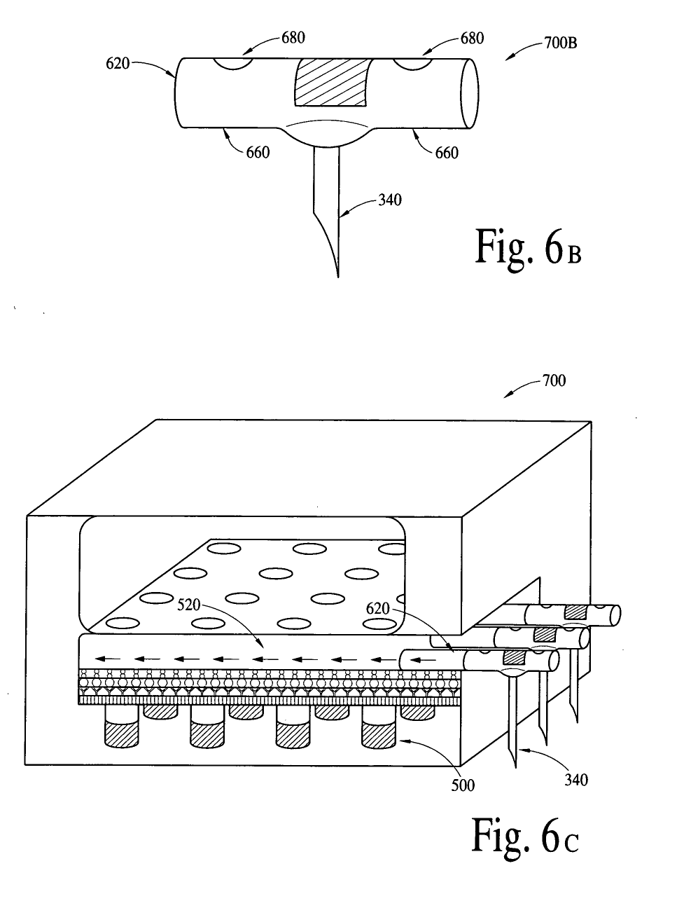

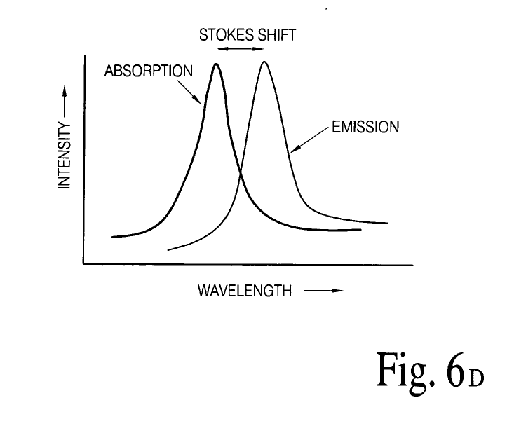

FIG. 6A illustrates the micropump 380, which can be continuously programmed (electronically and/or wirelessly) in a closed feedback loop to deliver the bioactive compounds 100 and/or bioactive molecules 100A based on a labeled (or label free) measurement of a disease specific biomarker 460 (in a patient's blood 440, which is propagated through a microfluidic channel 620 (the microfluidic channel 620 placed on a v-groove 640) to a microfluidic cavity 520), utilizing a receptor 240C, a fluorophore 120B, a biomolecular interface layer 480, a 2-D photonic crystal cavity (of both low and high index materials) 500, a microfluidic cavity 520, an optical beam diffuser 540, an optical beam splitter 560, a laser (e.g., MEMS enabled wavelength-tunable vertical cavity surface emitting) 580 and a spectrophotometer (e.g., a planar lightwave circuit/echelle gratings) 600.

Incident light from the laser 580 is split through the optical beam splitter 560 (the incident light is measured by the spectrophotometer 600 as a reference), then absorbed by the fluorophore 120B, attached to a disease specific biomarker 460, on the receptor 240C, on the biomolecular interface layer 480, on the 2-D photonic crystal cavity 500 and then the emitted fluorescence wavelength from the fluorophore(s) 120B attached to a disease specific biomarker 460, on the receptor 240C, on the biomolecular interface layer 480, on the 2-D photonic crystal cavity 500 is measured by the spectrophotometer 600.

The Stokes Shift is the difference between the absorption wavelength and fluorescence emission wavelength by the fluorophore 120B. The Stokes Shift can be utilized to detect a presence of a specific disease.

There is a predictable correlation between the physical diameter (e.g., from 5 nm to 10 nm) of a quantum dot/3-D photonic crystal fluorophore 120B and the fluorescence emission wavelength by the fluorophore 120B. This predictable correlation can be utilized to detect the simultaneous multi-color fluorescence from many biomarkers related to a specific disease (e.g., Alzheimer's) and/or multiple diseases (e.g., Alzheimer's and Cardiovascular).

FIG. 6B illustrates a MEMS module to draw blood from the patient into the microfluidic cavity 520, utilizing the microneedle 340, monolithically integrated with a micromachined (voltage deflectable) membrane 660, a membrane sensor 680 and a microfluidic channel 620. The microneedle 340 can be electrically powered and programmed to draw the patient's blood at a periodic interval of time.

Furthermore, the above MEMS module can consists of an array of: micro needles 340, micromachined membranes 660, membrane sensors 680 and microfludic channels 620.

To enable a detachable/removable MEMS module to draw the patient's blood; an array of microfluidic channels 620, placed on an array of precise silicon/ceramic v-groves 640, within a precisely machined connector, can be utilized.

FIG. 6C illustrates an integrated 2-D photonics crystal enabled diagnostic biomodule 700.

FIG. 6D illustrates the Stokes Shift (difference between the absorption wavelength and the fluorescence emission wavelength) due to a disease specific biomarker 460, on the receptor 240C, on the biomolecular interface layer 480, on the 2-D photonic crystal cavity 500.

### [Electrical Diagnostics Module for Detection of a Disease Specific Biomarker]

The micropump 380 can be continuously programmed (electronically and/or wirelessly) in a closed feedback loop to deliver the bioactive compounds 100 and/or bioactive molecules 100A based on the Stokes Shift.

Alternatively, the micropump 380 can be continuously programmed (electronically and/or wirelessly) in a closed feedback loop to deliver the bioactive compounds 100 and/or bioactive molecules 100A based on change in electrical characteristics of a graphene field effect transistor (FET) due to a disease specific biomarker 460, on the receptor 240C, on the biomolecular interface layer 480, on the single layer graphene 820.

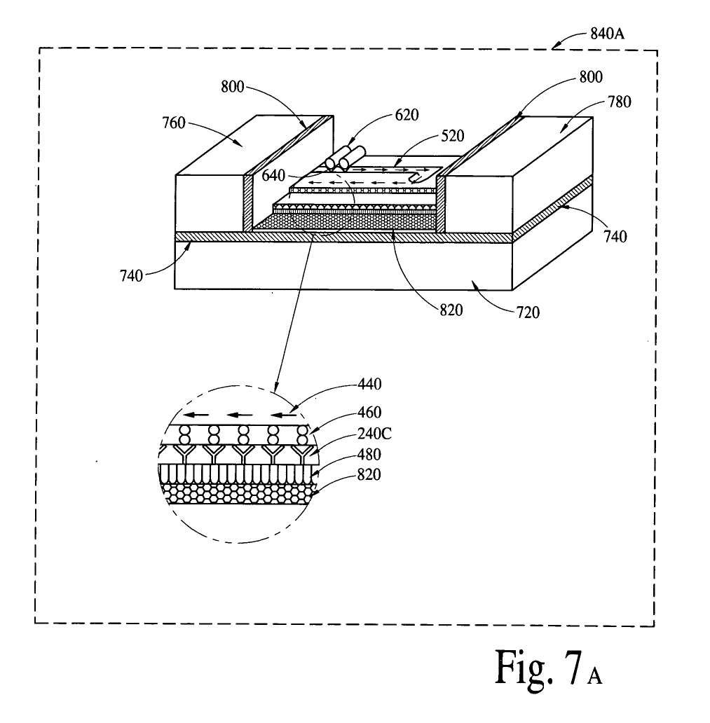

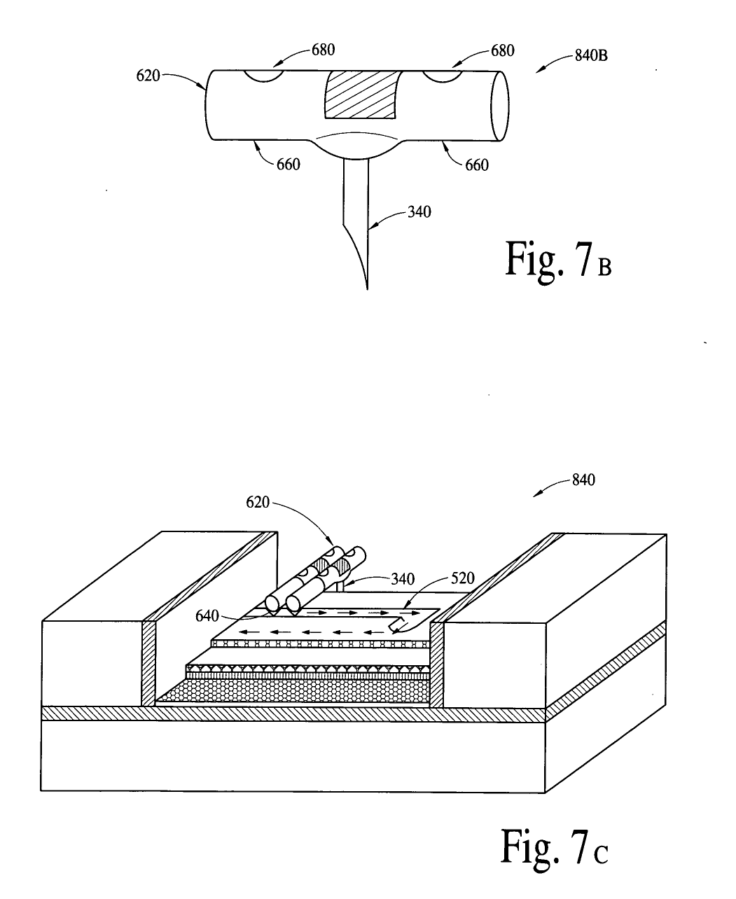

FIG. 7A illustrates graphene (a one-atom-thick layer of graphite carbon) enabled bio-field-effect transistor (FET): a semiconductor substrate 720, a gate oxide insulator thin-film 740, a source metal thin-film 760, a drain metal thin-film 780, a polymeric insulator thin-film 800, a single layer graphene 820, a disease specific biomarker 460 (in a patient's blood 440, which is propagated through the microfluidic channel 620 (the microfluidic channel 620 placed on the v-groove 640) to the microfluidic cavity 520), on the receptor 240C, on the biomolecular interface layer 480.

FIG. 7B illustrates a MEMS module to draw blood from the patient into the microfluidic cavity 520, utilizing the microneedle 340, monolithically integrated with a micromachined (voltage deflectable) membrane 660, a membrane sensor 680 and a microfluidic channel 620. The microneedle 340 can be electrically powered and programmed to draw the patient's blood at a periodic interval of time.

Furthermore, the above MEMS module can consists of an array of: micro needles 340, micromachined membranes 660, membrane sensors 680 and microfludic channels 620.

To enable a detachable/removable MEMS module to draw the patient's blood, an array of microfluidic channels 620, placed on an array of precise silicon/ceramic v-groves 640, within a precisely machined connector, can be utilized.

FIG. 7C illustrates an integrated graphene enabled diagnostics biomodule 840.

### [Closed Feedback Loop Subsystem for Active Delivery of Bioactive Compounds &/or Bioactive Molecules & Simultaneous Detection of a Disease Specific Biomarker]

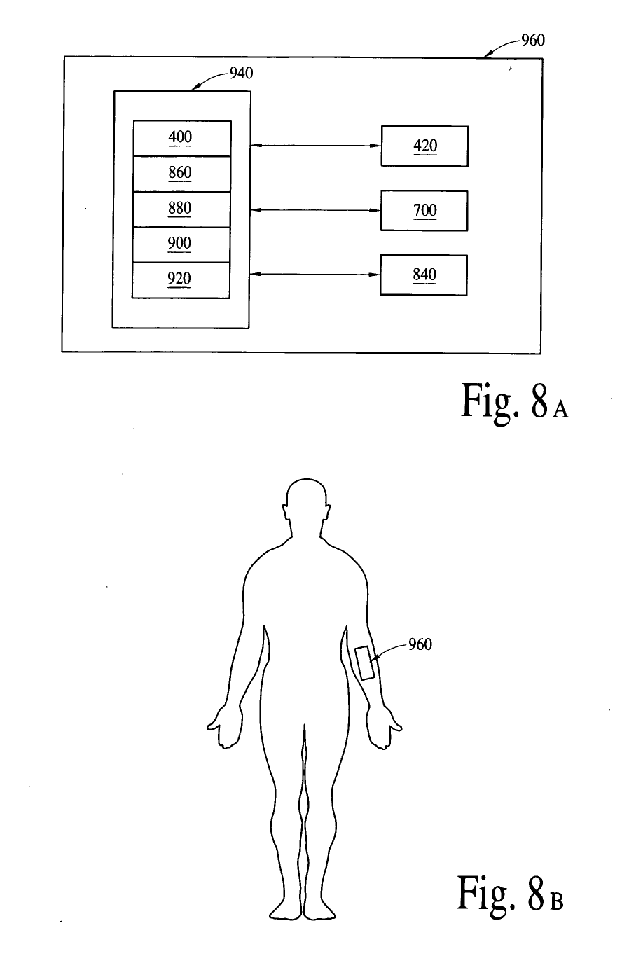

FIG. 8A illustrates a bioelectronics subsystem 960: integrating (a) a MEMS biomodule 420, (b) a 2-D photonic crystal enabled diagnostics biomodule 700, (c) a graphene enabled diagnostics biomodule 840 and (d) an electronic module 940.

The electronic module 940 includes at least an electrical power component 400, a microprocessor component 860, a memory/data storage component 880, a low-power wireless communication component 900 and an embedded operating algorithm 920, which can further interact with an intelligent expert diagnostic algorithm of diseases at a remote/cloud server.

FIG. 8B illustrates a real-life application of a bioelectronics subsystem 960.

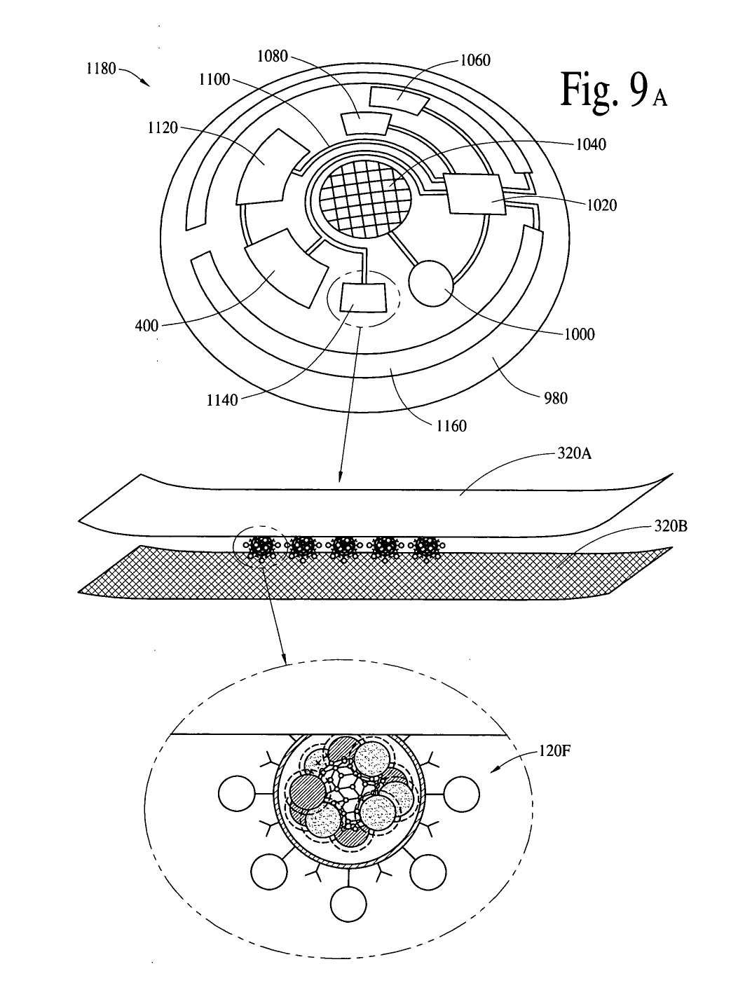

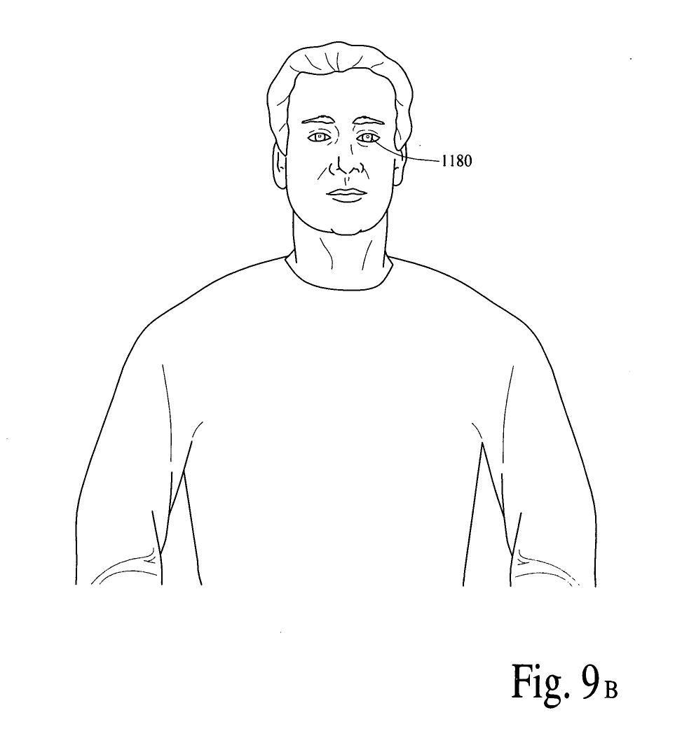

FIG. 9A illustrates a smart contact lens module 1180 of a biocompatible material (e.g., silicon/SU-8 resin/liquid crystal polymers/parylene/polyimide) 980, which integrates a control circuitry component 1000, a radio component 1020, an optical component (an array of microlens and/or quantum dot displays) 1040, a biosensor read-out component 1060, a biosensor component 1080, a solar cell component 1120, a micro patch component 1140 (for actively delivering the bioactive compounds 100 and/or bioactive molecules 100A based on the measurements of the bio-sensor read-out component 1060), an antenna component 1160 and an electrical powering component (a thick-film/thin-film/printed battery) 400 utilizing an electrical contact 1100.

Furthermore, the micro patch component 1140 can include a MEMS reservoir to store 120Fs.

The radio component 1020 is utilized for communicating (wirelessly) a disease condition analyzed by the biosensor read-out component 1060 (when a disease is detected by the biosensor component 1080).

An array of multi-wavelength (blue, green and red) quantum dot displays can be constructed as follows: optically pumps different-sized photonic crystals, whereas the photonic crystals can individually emit blue, green and red light based on their inherent sizes.

An optical pump can be generated from an optical emission by an electrical activation of semiconductor quantum-wells. Blue, green and red light can be multiplexed/combined to generate an array of quantum dot displays. The semiconductor quantum-wells are sandwiched between indium tin oxide (ITO) transparent front electrode and metal (e.g., aluminum or silver) back electrode.

An array of quantum dot displays can be manufactured by a contact printing process on a rigid (e.g., glass) or a flexible (e.g., plastic/graphene) substrate.

The solar cell component can be either a semiconductor (e.g., silicon) or a dye-sensitized based. A dye-sensitized solar cell consists of about two (2) micron thick meso-porous titanium oxide semi-conductor thin-film. This titanium oxide thin-film is coated with many types of light-absorbing organic dye molecules (e.g., porphyrins and phthalocyanines) embedded in nano crystals (e.g., 120A in FIG. 4C). Such a titanium oxide thin-film is immersed in an ionic electrolyte solution and is further sandwiched between two electrodes: indium tin oxide transparent front electrode and metal back electrode—where metal (e.g., aluminum or silver) back electrode has nano-corrugated plasmonic reflectors to trap more sunlight inside the solar cell.

Furthermore, the electrodes are deposited/printed and etched on glass and/or plastic substrate. Sunlight, through indium tin oxide transparent front electrode striking many light-absorbing organic dye molecules, frees negative charged electrons and creates positive charged “holes”, where the electrons are lost. The semi-conducting titanium dioxide particles collect the electrons and transfer them to an external circuit, producing an electric current.

FIG. 9B illustrates a real-life application of a smart retinal contact lens.

### [Summary of Products & Methods]

Definition: Component means any one of the following: a bioactive compound, a bioactive molecule, a functional molecule, a fluorophore, an electrical charge, an electronic component, an optical component and an algorithm.

[A] A neuro-protective dietary supplement includes: a) components of: Bacopa monnieri, Camellia sinensis, Cinnamomum zeylanicum, Curcuma longa, Evolvulus alsinoide, Mucuna pruriens and Withania somnifera.

Furthermore, the above neuro-protective dietary supplement includes: one or more components of: Hypericum perforatum, Nigella sativa/kalonji, Paeoniae alba and Salvia miltiorrhiza.

Furthermore, the above neuro-protective dietary supplement includes: one or more components of: Aronia melanocarpa, Citrus limonum, Daucus carota, Hibiscus spp., Malus domestica, Ribes nigrum, Sambucus nigra and Vaccinium spp.

Furthermore, the above neuro-protective dietary supplement includes: one or more components of: caffeine, citicoline, creatine and D-Ribose.

Furthermore, the above neuro-protective dietary supplement includes: one or more components of: acetyl-L-carnitine, coenzyme Q₁₀, lipoic acid, melatonin, theanine and uric acid.

Furthermore, the above neuro-protective dietary supplement includes: one or more curcumin-derived synthetic molecules of: FLLL-11, FLLL-12, GO-Y030 and GO-Y031.

Furthermore, the above neuro-protective dietary supplement includes: one or more components of: ebselen (or glutathione or N-acetyl-L-cysteine) and nano encapsulated superoxide dismutase (SOD).

Furthermore, the above neuro-protective dietary supplement includes: one or more components of: pterostilbene, quercetin and resveratrol.

Furthermore, the above neuro-protective dietary supplement includes: one or more components of: a mineral and a vitamin.

[B] A cardiovascular disease risk reducing dietary supplement includes: Crataegus oxyacantha, Inula racemosa, Irvingia gabonensis and Terminalia arjuna.

Furthermore, the above cardiovascular disease risk reducing dietary supplement includes: one or more components of: Aronia melanocarpa, Citrus limonum, Daucus carota, Hibiscus spp., Malus domestica, Ribes nigrum, Sambucus nigra and Vaccinium spp.

Furthermore, the above cardiovascular disease risk reducing dietary supplement includes: one or more components of: ebselen (or glutathione or N-acetyl-L-cysteine) and nano encapsulated superoxide dismutase (SOD).

Furthermore, the above cardiovascular disease risk reducing dietary supplement includes: one or more components of: plant sterol, pterostilbene, quercetin and resveratrol.

Furthermore, the above cardiovascular disease risk reducing dietary supplement includes: one or more components of a mineral and a vitamin.

[C] A Type-2 Diabetes disease risk reducing dietary supplement includes: a) Touchi extract b) components of: Coccinia indica, Irvingia gabonensis, Momordica charantia and Salacia oblonga and c) acetyl-L-carnitine, beta glucan, coenzyme Q₁₀, lipoic acid and nobiletin (or naringenin).

Furthermore, the above Type-2 Diabetes disease risk reducing dietary supplement includes: one or more components of: Andrographis paniculata, Artemisia princeps and Nigella sativa/kalonji.

Furthermore, the above Type-2 Diabetes disease risk reducing dietary supplement includes: one or more components of: Camellia sinensis, Euterpe oleracea, Hippophae rhamnoides, Lycium barbarum, Phyllanthus emblica, Punica granatum and Vitis spp.

Furthermore, the above Type-2 Diabetes disease risk reducing dietary supplement includes: one or more components of: Aronia melanocarpa, Citrus limonum, Daucus carota, Hibiscus spp., Malus domestica, Ribes nigrum, Sambucus nigra and Vaccinium spp.

Furthermore, the above Type-2 Diabetes disease risk reducing dietary supplement includes: one or more components of: ebselen (or glutathione or N-acetyl-L-cysteine) and nano encapsulated superoxide dismutase (SOD).

Furthermore, the above Type-2 Diabetes disease risk reducing dietary supplement includes: one or more components of: pterostilbene, quercetin, resveratrol and sulforaphane.

Furthermore, the above Type-2 Diabetes disease risk reducing dietary supplement includes: one or more components of: a mineral and a vitamin.

[D] A dietary super antioxidant dietary supplement includes: any three components of a) Actinidia chinenesis, Ananas comosus, Cocos nucifera, Garcinia mangostana, Litchi chinensis and Vitis spp. b) components of: Aronia melanocarpa, Citrus limonum, Daucus carota, Hibiscus spp., Malus domestica, Ribes nigrum, Sambucus nigra and Vaccinium spp. c) coenzyme Q₁₀.

Furthermore, the above dietary super antioxidant dietary supplement includes: one or more components of: ebselen (or glutathione or N-acetyl-L-cysteine) and nano encapsulated superoxide dismutase (SOD).

Furthermore, the above dietary super antioxidant dietary supplement includes: one or more components of: citicoline, D-Ribose, L-analyl-L-glutamine and theanine.

Furthermore, the above dietary super antioxidant dietary supplement includes: one or more components of: plant sterol, pterostilbene, quercetin and resveratrol.

Furthermore, the above dietary super antioxidant dietary supplement includes: one or more components of: a mineral and a vitamin.

[E] A dietary sweetener includes: erythritol and Stevia rebaudiana.

The above sweetener further includes: one or more components of: Capparis masaikai.

The above sweetener further includes: one or more components of: Curculigo latifolia.

The above sweetener further includes: one or more components of: Dioscoreophyllum cumminsii.

The above sweetener further includes: one or more components of: Momordica/Siraitia grosvenorii.

The above sweetener further includes: one or more components of: Pentadiplandra brazzeana.

The above sweetener further includes: one or more components of: Synsepalum dulcificum.

The above sweetener further includes: one or more components of: Thaumatococcus daniellii.

[F] A nano assembly (as in FIG. 4F) with curcumin and/or curcumin-derived synthetic molecules (FLLL-11, FLLL-12, GO-Y030 and GO-Y031) can be an effective therapy against cancer (including brain cancer).

[G] A nano assembly (as in FIG. 4F) with acetyl-L-carnitine, curcumin, curcumin-derived synthetic molecules (FLLL-11, FLLL-12, GO-Y030 and GO-Y031), L-DOPA and melatonin can cross blood brain barrier (BBB) and can be an effective therapy against neuro-degenerative diseases (e.g., Alzheimer's and/or Parkinson's disease).

[H] A nano assembly (as in FIG. 4F) with luric acid can be an effective therapy against acne.

[I] A nano assembly (as in FIG. 4F) with nitrous oxide can be an effective therapy against erectile dysfunction.

[J] A nano assembly (as in FIG. 4F) with micro-RNA (mi-RNA) or small interfering RNA (si-RNA) can be an effective therapy against various diseases.

[K] A long-term micro patch includes: a) a nano crystal for nano assembling of bioactive compounds and/or bioactive molecules, b) a MEMS reservoir for storing the nano crystal with the bioactive compound and/or the bioactive molecule, c) a thin-film for attaching with the MEMS reservoir and d) a bioactive compound and/or a bioactive molecule.

[L] A short-term micro patch includes: a) a nano crystal for nano assembling of bioactive compounds and/or bioactive molecules, b) a thin-film for attaching with the nano crystals and c) a bioactive compound and/or a bioactive molecule.

[M] A passive method of delivering the bioactive compounds and/or bioactive molecules, which utilizes a MEMS reservoir with monolithically integrated microneedles.

[N] An active method of delivering the bioactive compounds and/or bioactive molecules, which utilizes a MEMS reservoir with monolithically integrated microneedles, where the MEMS reservoir further includes: an electrically powered micropump.

[O] A bioelectronics subsystem includes: a) a MEMS based blood drawing module, b) a photonic-crystal based optical diagnostic module, c) a graphene based electrical diagnostics module and d) a MEMS based active delivery module.

Furthermore the above bioelectronics subsystem includes: a) a needle for penetrating into skin (e.g., the skin of a human body) for collecting blood, b) a fluidic channel for guiding blood onto a biomolecular interface layer, c) the biomolecular interface layer for interacting with a biomarker and a receptor, d) the receptor for binding with the biomarker, e) a photonic crystal structure for a change in optical characteristics due to an interaction of the biomarker with the receptor on the biomolecular layer, 1) a laser for incident light onto the photonic crystal structure, g) a device for measuring the change in optical characteristics, h) a graphene field-effect transistor (FET) for measuring a change in electrical characteristics due to an interaction of the biomarker with the receptor on the biomolecular layer, i) a bioactive compound and/or a bioactive molecule, j) a MEMS reservoir for storing the bioactive compound and/or the bioactive molecule and k) a pump for propagating the bioactive compound an/or the bioactive molecule through the skin.

Furthermore the above bioelectronics subsystem includes: one or more components of: an electrical charge, a fluorophore, an immune surveillance evading functional molecule, a nano crystal and a targeting ligand molecule.

Furthermore the above bioelectronics subsystem includes: one or more components of: an optical beam diffuser, an optical beam splitter, a voltage controlled membrane and a v-groove.

Furthermore the above bioelectronics subsystem includes: one or more components of: a microprocessor component, a memory/data storage component, a wireless communication component, an electrical powering component and an algorithm.

Furthermore the above bioelectronics subsystem utilizes: an intelligent expert diagnostic algorithm of diseases at a remote/cloud server.

[P] A retinal contact lens includes: a micro patch (as mentioned in K or L) for delivering the bioactive compounds and/or bioactive molecules.

[Q] The above retinal contact lens further includes: a biosensor component and a biosensor read-out component.

[R] The above retinal contact lens further includes: an optical component (an array of microlens and/or quantum dot displays).

[S] The above retinal contact lens further includes: a solar cell component.

[T] The retinal contact lens in [R] has many types of light-absorbing dye molecules (e.g., porphyrins and phthalocyanines) embedded in nano crystals.

[U] The above retinal contact lens further includes: a battery (thin-film/printed) component.

[V] The above retinal contact lens further includes: a radio component.

[W] The above retinal contact lens further includes: an antenna component.

The above disclosed descriptions are only the most preferred embodiments of the present invention. However it is not intended to be limiting. Numerous variations and/or modifications are possible within the scope of the present invention.

I claim:

1. A dietary supplement, comprising: a) components of: Bacopa monnieri, Camellia sinensis, Cinnamomum zeylanicum, Curcuma longa, Evolvulus alsinoide, Mucuna pruriens and Withania somnifera.

2. A dietary supplement, as in claim 1, further comprising: one or more components, selected from the group consisting of: Hypericum perforatum, Nigella sativa/kalonji, Paeoniae alba and Salvia miltiorrhiza.

3. A dietary supplement, as in claim 1, further comprising: one or more components, selected from the group consisting of: Aronia melanocarpa, Citrus limonum, Daucus carota, Hibiscus spp., Malus domestica, Ribes nigrum, Sambucus nigra and Vaccinium spp.

4. A dietary supplement, as in claim 1, further comprising: one or more components, selected from the group consisting of: caffeine, citicoline, creatine and D-ribose.

5. A dietary supplement, as in claim 1, further comprising: one or more components, selected from the group consisting of: acetyl-L-carnitine, coenzyme Q₁₀, lipoic acid, melatonin, theanine and uric acid.

6. A dietary supplement, as in claim 1, further comprising: one or more molecules, selected from the group consisting of: FLLL-11, FLLL-12, GO-Y030 and GO-Y031

7. A dietary supplement, as in claim 1, further comprising: one or more components, selected from the group consisting of: ebselen (or glutathione or n-acetyl-L-cysteine) and superoxide dismutase (SOD).

8. A dietary supplement, as in claim 1, further comprising: one or more components, selected from the group consisting of: pterostilbene, quercetin and resveratrol.

9. A dietary supplement, as in claim 1, further comprising: one or more components, selected from the group consisting of: a mineral and a vitamin.

10. A dietary supplement, comprising: Crataegus oxyacantha, Inula racemosa, Irvingia gabonensis and Terminalia arjuna.

11. A dietary supplement, as in claim 10, further comprising: one or more components, selected from the group consisting of: Aronia melanocarpa, Citrus limonum, Daucus carota, Hibiscus spp., Malus domestica, Ribes nigrum, Sambucus nigra and Vaccinium spp.

12. A dietary supplement, as in claim 10, further comprising: one or more components, selected from the group consisting of: ebselen (or glutathione or n-acetyl-L-cysteine) and superoxide dismutase (SOD).

13. A dietary supplement, as in claim 10, further comprising: one or more components, selected from the group consisting of: plant sterol, pterostilbene, quercetin and resveratrol.

14. A dietary supplement, as in claim 10, further comprising: one or more components, selected from the group consisting of: a mineral and a vitamin.

15. A dietary supplement, comprising: a) Touchi extract; b) components of Coccinia indica, Irvingia gabonensis, Momordica charantia and Salacia oblonga; and c) components of acetyl-L-carnitine, beta glucan, coenzyme Q₁₀, lipoic acid and naringenin (or nobiletin).

16. A dietary supplement, as in claim 15, further comprising: one or more components, selected from the group consisting of: Andrographis paniculata, Artemisia princeps and Nigella sativa/kalonji.

17. A dietary supplement, as in claim 15, further comprising: one or more components, selected from the group consisting of: Camellia sinensis, Euterpe oleracea, Hippophae rhamnoides, Lycium barbarum, Phyllanthus emblica, Punica granatum and Vitis spp.

18. A dietary supplement, as in claim 15, further comprising: one or more components, selected from the group consisting of: Aronia melanocarpa, Citrus limonum, Daucus carota, Hibiscus spp., Malus domestica, Ribes nigrum, Sambucus nigra and Vaccinium spp.

19. A dietary supplement, as in claim 15, further comprising: one or more components, selected from the group consisting of ebselen (or glutathione or n-acetyl-L-cysteine) and superoxide dismutase (SOD).

20. A dietary supplement, as in claim 15, further comprising: one or more components, selected from the group consisting of: pterostilbene, quercetin, resveratrol and sulforaphane.

21. A dietary supplement, as in claim 15, further comprising: one or more components, selected from the group consisting of: a mineral and a vitamin.
